(19) United States
(12) Patent Application Publication
(10) Pub. No.: US 2024/0058350 A1
(43) Pub. Date: Feb. 22, 2024

(54) Methods of Treatment

(71) Applicant: Arena Pharmaceuticals, Inc., New York, NY (US)

(72) Inventors: John William Adams, La Jolla, CA (US); Prodromos Anthopoulos, Hunenberg (CH); Kathe Lynn Stauber, San Diego, CA (US); Brandon James Walsh, Carlsbad, CA (US)

(73) Assignee: Arena Pharmaceuticals, Inc., New York, NY (US)

(21) Appl. No.: 18/259,843

(22) PCT Filed: Dec. 30, 2021
(86) PCT No.: PCT/US2021/065777

§ 371 Date: Jun. 29, 2023

Related U.S. Application Data
(60) Provisional application No. 63/133,160, filed on Dec. 31, 2020.

(51) Int. Cl. A61K 31/5377; A61K 9/00; A61P 7/02

(52) U.S. Cl. CPC A61K 31/5377; A61K 9/0053; A61P 7/02; A61K 9/0019

(57) ABSTRACT

Provided are methods for the parenteral use of 3-methoxy-N-[3-(2-methylpyrazol-3-yl)-4-(2-morpholin-4-ylethoxy)phenyl]benzamide, or a pharmaceutically acceptable salt, hydrate, or solvate thereof, and the sequential use of parenteral and oral formulations of 3-methoxy-N-[3-(2-methylpyrazol-3-yl)-4-(2-morpholin-4-ylethoxy)phenyl]benzamide, or a pharmaceutically acceptable salt, hydrate, or solvate thereof.

Cardiovascular disease (CVD) is the leading cause of death globally. Among CVD, coronary artery disease (CAD) is the single most common cause of death, resulting in 1 in every 4 deaths in the US. The main cause of CAD is atherosclerosis, an inflammatory disease characterized by focal thickenings of the arterial intima containing lipids, connective tissue, and various cell types. Atherosclerotic plaque buildup progressively narrows the lumen of coronary arteries and leads to clinical symptoms such as angina. Highly inflammatory and unstable plaques (vulnerable plaques) may eventually rupture and lead to potentially fatal events such as myocardial infarction (MI). In both cases, the prevention of oxygen delivery to the myocardium through impaired or blocked coronary blood flow gives rise to clinical events.

Significant advances have been made in medical therapy to prevent or treat CAD; however, because the disease is often detected in a late stage and difficult to reverse, a large percentage of patients remain symptomatic or develop complications and require invasive, mechanical interventions such as PCI or coronary artery bypass graft (CABG). Despite broad implementation of primary PCI and an improvement in “door-to-balloon” times, corresponding improvement in mortality has plateaued in recent years. Notably, a significant proportion of patients undergoing PCI, mainly for treatment of ACS, fail to achieve full myocardial or microcirculatory reperfusion despite resolution of the epicardial coronary occlusion. This is sometimes expressed angiographically as slow/no-reflow, but even in cases with normal epicardial flow, microvascular obstruction (MVO) can detrimentally affect microcirculatory perfusion and has been reported in approximately 40-60% of patients following PCI for ST-elevation myocardial infarction (STEMI).

MVO can be assessed immediately following PCI using several invasive techniques, including angiographic assessment of the myocardial blush grade (MBG) and assessment of coronary physiology, based on parameters such as the index of microcirculatory resistance (IMR). Cardiac magnetic resonance (CMR) imaging, considered to be the ‘gold standard’ for diagnosis of MVO, can be performed in the days following PCI. Regardless of the method used, the presence of MVO has been associated with worse clinical outcomes, including mortality and hospitalization for heart failure, related to suboptimal cardiac function and recovery in the days and months following the PCI procedure. Several treatment options have been explored, including the use of vasodilators and anti-platelet agents; however, clinical efficacy data from these interventions is limited and currently no treatment has been demonstrated to be beneficial for prevention or treatment of MVO in the setting of PCI. Therefore, there is an unmet need for safe and effective agents that improve post-PCI cardiac functional recovery and clinical outcomes through the prevention and treatment of MVO.

### SUMMARY

Described herein are the first clinical studies evaluating a 5-HT2A modulator for microvascular obstruction, and the first clinical studies evaluating intravenous (IV) administration of 3-methoxy-N-[3-(2-methylpyrazol-3-yl)-4-(2-morpholin-4-ylethoxy)phenyl]benzamide (Compound 1).

The methods of administration described herein provide new options for an individual undergoing PCI, including fixed sequence dosing with Compound 1 that provides for continuing therapeutic exposure during a PCI procedure and the following recovery period.

Provided herein is a method of treating or preventing microvascular obstruction in an individual in need thereof, comprising administering parenterally a formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to the individual.

In some embodiments, the method further comprises subsequently administering orally a formulation of Compound 1, or a pharmaceutically acceptable salt, hydrate, or solvate thereof, to the individual for a second period of time, where the parenterally administered formulation is administered for a first period of time.

Also provided is a method for administering 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to an individual in need thereof, comprising administering parenterally a formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to the individual for a first period of time; and subsequently administering orally a formulation of Compound 1, or a pharmaceutically acceptable salt, hydrate, or solvate thereof, to the individual for a second period of time.

Also provided is a method for preserving vascular integrity in an individual in need thereof, comprising administering parenterally a formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to the individual for a first period of time; and subsequently administering orally a formulation of Compound 1, or a pharmaceutically acceptable salt, hydrate, or solvate thereof, to the individual for a second period of time.

Also provided is a method for the prevention of a major adverse cardiovascular event (MACE) by preventing and/or treating microvascular obstruction (MVO) in an individual undergoing percutaneous coronary intervention (PCI) comprising administering parenterally a formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to the individual for a first period of time; and subsequently administering orally a formulation of Compound 1, or a pharmaceutically acceptable salt, hydrate, or solvate thereof, to the individual for a second period of time.

Also provided is a method for administering 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to an individual in need thereof, comprising administering parenterally 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to the individual, comprising administering intravenously a formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to the individual, and administering by infusion a formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to the individual.

In some embodiments, the method further comprises subsequently administering orally a formulation of Compound 1, or a pharmaceutically acceptable salt, hydrate, or solvate thereof, to the individual for a second period of time, where the parenterally administered formulations are administered for a first period of time.

Also provided is a kit comprising a parenteral formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof; an oral formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof; and instructions indicating the medication is for sequential use to prevent microvascular obstruction, to preserve vascular integrity, and/or to prevent a major adverse cardiovascular event (MACE) by preventing and/or treating microvascular obstruction (MVO) in an individual undergoing percutaneous coronary intervention (PCI).

Also provided is a pharmaceutical formulation for parenteral administration comprising a sterilized solution comprising about 5 mg/mL to about 25 mg/mL (adjusted free-base concentration) of Compound 1, or a pharmaceutically acceptable salt, hydrate, or solvate thereof.

Also provided is a method of safely administering 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to an individual in need thereof, comprising discontinuing administration of an inhibitor or inducer of CYP3A4, CYP3A5 and/or P-glycoprotein, or a proton pump inhibitor, to the individual; not co-administering an inhibitor or inducer of CYP3A4, CYP3A5 and/or P-glycoprotein, or a proton pump inhibitor, to the individual; co-administering a reduced dose of an inhibitor or inducer of CYP3A4, CYP3A5 and/or P-glycoprotein, or a proton pump inhibitor, to the individual; or administering a reduced dose of the Compound 1, or a pharmaceutically acceptable salt, hydrate, or solvate thereof to the individual, when co-administered with an inhibitor or inducer of CYP3A4, CYP3A5 and/or P-glycoprotein, or a proton pump inhibitor; or administering a lower dose of the Compound 1, or a pharmaceutically acceptable salt, hydrate, or solvate thereof and a lower dose of an inhibitor or inducer of CYP3A4, CYP3A5 and/or P-glycoprotein, or a proton pump inhibitor, to the individual.

In some embodiments, the Compound 1, or a pharmaceutically acceptable salt, hydrate, or solvate thereof, is a pharmaceutically acceptable salt of Compound 1.

In some embodiments, the Compound 1, or a pharmaceutically acceptable salt, hydrate, or solvate thereof, is the HCl salt of Compound 1.

These and other aspects of the invention will be apparent upon reference to the following detailed description. To this end, various references are set forth herein which describe in more detail certain background information, procedures, compounds, and/or compositions, and are each hereby incorporated by reference in their entirety.

### DETAILED DESCRIPTION

As used in the present specification, the following words and phrases are generally intended to have the meanings as set forth below, except to the extent that the context in which they are used indicates otherwise.

COMPOUND 1: As used herein, “Compound 1” refers to 3-methoxy-N-[3-(2-methylpyrazol-3-yl)-4-(2-morpholin-4-ylethoxy)phenyl]benzamide, including crystalline forms thereof. As a non-limiting example, Compound 1 may be present in the crystalline form disclosed in U.S. Pat. No. 8,980,891 (incorporated by reference herein in its entirety), which may be characterized by one or more of the following ° 20 values for the peaks in the PXRD spectrum with CuKα radiation: 5.0998, 18.7064, 19.1157, 19.3029, and 23.6567, wherein the reported peaks can vary by about 0.2° 20.

Compound 1, or a pharmaceutically acceptable salt, hydrate, or solvate thereof, and its active metabolites (M1 and M2) have a high affinity for human 5-HT2A with no appreciable affinity for 5-HT_{2B} and 5-HT_{2C} receptors. M1 refers to N-(4-(2-(2-hydroxyethylamino)ethoxy)-3-(1-methyl-1H-pyrazol-5-yl)phenyl)-3-methoxybenzamide. M2 refers to N-(4-(2-aminoethoxy)-3-(1-methyl-1H-pyrazol-5-yl)phenyl)-3-methoxybenzamide. Compound 1, or a pharmaceutically acceptable salt, hydrate, or solvate thereof, inhibits serotonin-mediated vasoconstriction and inhibits serotonin-mediated amplification of platelet aggregation.

ACUTE CORONARY SYNDROME: As used herein, “acute coronary syndrome” or “ACS” refers to three types of coronary artery disease that are associated with sudden rupture of plaque inside the coronary artery, including unstable angina (UA), non-ST segment elevation myocardial infarction (NSTEMI), and ST segment elevation myocardial infarction (STEMI). The location of the blockage, the length of time that blood flow is blocked and the amount of damage that occurs determines the type of acute coronary syndrome. Myocardial infarction is a major cause of death and disability worldwide. In the United States alone, more than one million hospitalizations are required for ACS of which more than half are for acute myocardial infarction (AMI) including roughly two-thirds with NSTEMI and the rest with STEMI.

PERCUTANEOUS CORONARY INTERVENTION: As sued herein, the term “percutaneous coronary intervention” or “PCI” means coronary angioplasty which is a therapeutic procedure to treat the stenotic (narrowed) coronary arteries of the heart found in coronary heart disease.

GLOBAL REGISTRY OF ACUTE CORONARY EVENTS SCORE: As used herein, the term “Global Registry of Acute Coronary Events (GRACE) score,” “Global Registry of Acute Coronary Events score,” “GRACE risk score,” or “GRACE score” refers to a method of identifying higher risk individuals. Using the GRACE risk score, eight factors independently predict risk of heart attack and/or death: age, heart rate, systolic blood pressure, renal function, congestive heart failure, ST-segment deviation, cardiac arrest, and elevated biomarker.

SEQUENTIAL THERAPY: As used herein, “sequential therapy” refers to the use of parenteral administration (e.g., intravenous injection) for an initial stage of treatment, followed by oral administration after the clinical symptoms are stable and improved. Although intravenous drip is used for the purpose of timely treatment for individual who cannot be administrated orally, it will inevitably lead to corresponding adverse reactions, such as infusion reaction, vascular stimulation and phlebitis, causing pain to the individual. Sequential therapy can shorten the time of intravenous administration and reduce the incidence of adverse reactions related to infusion, which can greatly shorten the hospitalization time for an individual, save the expenses of individuals and medical institutions, save limited economic resources, and reduce the related social labor losses caused by hospitalization of individuals.

PARENTERAL: As used herein, “parenteral” drug administration means any non-oral means of administration, but is generally interpreted as relating to injecting directly into the body, bypassing the skin and mucous membranes. Common parenteral routes are intramuscular (IM), subcutaneous (SC) and intravenous (IV).

TREAT, TREATING, OR TREATMENT: As used herein the term “treat,” “treating,” or “treatment” refers to the administration of therapy to an individual (i.e., a human) who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with a particular disorder. Therefore, treating a disorder does not necessarily mean a reduction in severity of all symptoms associated with a disorder and does not necessarily mean a complete reduction in the severity of one or more symptoms associated with a disorder.

PREVENT, PREVENTING, OR PREVENTION: As used herein, the term “prevent,” “preventing” or “prevention” means prevention of the occurrence or onset of one or more symptoms associated with a particular disorder and does not necessarily mean the complete prevention of a disorder. For example, the term “prevent,” “preventing” and “prevention” refers to the administration of therapy on a prophylactic or preventative basis to an individual who may ultimately manifest at least one symptom of a disease or condition but who has not yet done so. Such individuals can be identified since risk factors that are known to correlate with the subsequent occurrence of the disease. Alternatively, prevention therapy can be administered without prior identification of a risk factor, as a prophylactic measure. Delaying the onset of the at least one symptom can also be considered prevention or prophylaxis.

DOSE: As used herein, a “dose” means the measured quantity of an active agent to be taken at one time by an individual. In certain embodiments, wherein the active agent is not the free base of Compound 1, the quantity is the molar equivalent to the corresponding amount of the free base of Compound 1. For example, often a drug is packaged in a pharmaceutically acceptable salt form, for example the HCl salt of Compound 1, and the dosage for strength refers to the mass of the molar equivalent of the corresponding free base of Compound 1.

TOLERATE: As used herein, an individual is said to “tolerate” a dose of a compound if administration of that dose to that individual does not result in an unacceptable adverse event or an unacceptable combination of adverse events. One of skill in the art will appreciate that tolerance is a subjective measure and that what may be tolerable to one individual may not be tolerable to a different individual. For example, one individual may not be able to tolerate headache, whereas a second individual may find headache tolerable but is not able to tolerate vomiting, whereas for a third individual, either headache alone or vomiting alone is tolerable, but the individual is not able to tolerate the combination of headache and vomiting (even if the severity of each is less than when experienced alone).

ADVERSE EVENT: As used herein, an “adverse event” is an untoward medical occurrence that is associated with treatment with Compound 1 or a pharmaceutically acceptable salt, solvate or hydrate thereof. In one embodiment, an adverse event is selected from dizziness, headache, and nausea.

It is understood that when the phrase “pharmaceutically acceptable salts, solvates and hydrates” or the phrase “pharmaceutically acceptable salt, solvate or hydrate” is used when referring to Compound 1, it embraces pharmaceutically acceptable solvates and/or hydrates of Compound 1, pharmaceutically acceptable salts of Compound 1, as well as pharmaceutically acceptable solvates and/or hydrates of pharmaceutically acceptable salts of Compound 1. It is also understood that when the phrase “pharmaceutically acceptable solvates and hydrates” or the phrase “pharmaceutically acceptable solvate or hydrate” is used when referring to Compound 1 that are salts, it embraces pharmaceutically acceptable solvates and/or hydrates of such salts.

It will be apparent to those skilled in the art that the dosage forms described herein may comprise, as the active component, either Compound 1 or a pharmaceutically acceptable salt or as a solvate or hydrate thereof. Moreover, various hydrates and solvates of Compound 1 and their salts will find use as intermediates in the manufacture of pharmaceutical compositions. Typical procedures for making and identifying suitable hydrates and solvates, outside those mentioned herein, are well known to those in the art. See. e.g., pp. 202-209 of K. J. Guillory. “Generation of Polymorphs, Hydrates, Solvates, and Amorphous Solids,” in: Polymorphism in Pharmaceutical Solids, ed. Harry G. Britain. Vol. 95. Marcel Dekker. Inc., New York, 1999. Accordingly, one aspect of the present disclosure pertains to methods of prescribing and/or administering hydrates and solvates of Compound 1 and/or its pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like.

As used herein, the term “greater than” is used interchangeably with the symbol > and the term less than is used interchangeably with the symbol <. Likewise, the term greater than or equal to is interchangeably with the symbol ≥, and the term less than or equal to is interchangeably with the symbol ≤.

When an integer is used in a method disclosed herein, the term “about” can be inserted before the integer.

Throughout this specification, unless the context requires otherwise, the word “comprise”, or variations such as “comprises” or “comprising” will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention(s) described herein is susceptible to variations and modifications other than those specifically described. It is to be understood that the invention(s) includes all such variations and modifications. The invention(s) also includes all of the steps, features, compositions and compounds referred to or indicated in this specification, individually or collectively, and any and all combinations or any two or more of said steps or features unless specifically stated otherwise.

The present invention(s) is not to be limited in scope by the specific embodiments described herein, which are intended for the purpose of exemplification only. Functionally equivalent products, compositions and methods are clearly within the scope of the invention(s), as described herein. For example, the dosage amounts of Compound 1 or pharmaceutically acceptable salts, solvates or hydrates thereof disclosed herein can be replaced with dosage amounts for other salt or crystalline forms, formulations, and dosage regimens that exhibit bioequivalence to the specified amount of Compound 1 or pharmaceutically acceptable salts, solvates or hydrates thereof, including forms with 80-125% of the AUC and/or C, as measured by methods disclosed in the FDA's Guidance for Industry for Bioavailability and Bioequivalence (Guidance for Industry: Bioavailability and Bioequivalence Studies for Orally Administered Drug Products-General Considerations. US. Department of Health and Human Services. Food and Drug Administration. Center for Drug Evaluation and Research (CDER). March 2003. Revision 1, www.fda.gov/cder/guidance/index.htm). For example, the dosage amounts of Compound 1 disclosed herein can be replaced with dosage amounts for other salt or crystalline forms, formulations, or dosage regimens that exhibit bioequivalence to that dosage amount of Compound 1.

It is appreciated that certain features of the invention(s), which are, for clarity, described in the context of separate embodiments, can also be provided in combination in a single embodiment. For example, a method that recites prescribing Compound 1 or a pharmaceutically acceptable salt, solvate or hydrate thereof and a separate method of the invention reciting administering Compound 1 or a pharmaceutically acceptable salt, solvate or hydrate thereof can be combined into a single method reciting prescribing and administering Compound 1 or a pharmaceutically acceptable salt, solvate or hydrate thereof.

### Methods of Use

Provided is a method for administering 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to an individual in need thereof, comprising:
administering parenterally a formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to the individual for a first period of time; and
subsequently administering orally a formulation of Compound 1, or a pharmaceutically acceptable salt, hydrate, or solvate thereof, to the individual for a second period of time.

Also provided is a method for preventing microvascular obstruction in an individual in need thereof, comprising:
administering parenterally a formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to the individual for a first period of time; and
subsequently administering orally a formulation of Compound 1, or a pharmaceutically acceptable salt, hydrate, or solvate thereof, to the individual for a second period of time.

Also provided is a method for preserving vascular integrity in an individual in need thereof, comprising:
administering parenterally a formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to the individual for a first period of time; and
subsequently administering orally a formulation of Compound 1, or a pharmaceutically acceptable salt, hydrate, or solvate thereof, to the individual for a second period of time.

Also provided is a method for the prevention of a major adverse cardiovascular event (MACE) by preventing and/or treating microvascular obstruction (MVO) in an individual with acute coronary syndrome (ACS) undergoing percutaneous coronary intervention (PCI) comprising:
administering parenterally a formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to the individual for a first period of time; and
subsequently administering orally a formulation of Compound 1, or a pharmaceutically acceptable salt, hydrate, or solvate thereof, to the individual for a second period of time.

In some embodiments, an individual's response to a parenteral formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof is used to determine the administration of an oral formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof. In some embodiments, a parenteral formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof is administered to an individual, and if a measurement of efficacy is observed, an oral formulation of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof is administered to the individual. In some embodiments, the measurement of efficacy is a serotonin level. In some embodiments, the measurement of efficacy is a troponin level.

In some embodiments, the individual is undergoing PCI for ACS.

In some embodiments, the ACS is non-ST-elevation myocardial infarction (NSTEMI).

In some embodiments, the individual has ACS.

In some embodiments, the individual has stable angina.

In some embodiments, the individual has unstable angina.

In some embodiments, the individual presents with STEMI.

In some embodiments, the individual presents with NSTEMI.

In some embodiments, the individual presents with high-risk NSTEMI.

In some embodiments, the individual has a high risk GRACE score. In some embodiments, the individual has a GRACE score >100, 105, 110, 115, 120, 125, 130, 135, 140, 145, or 150. In some embodiments, the individual has a GRACE score >140.

In some embodiments, the individual has an elevated risk of developing a MACE. In some embodiments, the MACE is selected from a nonfatal myocardial infarction, coronary intervention, and cardiac death. In some embodiments, the MACE is selected from cardiac death, myocardial re-infarction, clinically driven myocardial revascularization, and hospitalization with discharge diagnosis of heart failure or coronary artery disease.

In some embodiments, the individual has an elevated risk of developing MVO.

In some embodiments, the individual has an elevated risk of mortality 6 months post-PCI.

In some embodiments, the individual is undergoing PCI. In some embodiments, the individual is undergoing PCI for stable angina.

In some embodiments, the individual is undergoing PCI for unstable angina.

In some embodiments, the individual is undergoing PCI for STEMI.

In some embodiments, the individual is undergoing PCI for NSTEMI.

In some embodiments, the individual is undergoing PCI for high-risk NSTEMI.

In some embodiments, the individual is undergoing primary PCI.

In some embodiments, the individual is undergoing elective PCI.

In some embodiments, the individual is undergoing saphenous vein graft.

In some embodiments, the individual is undergoing a stent procedure.

In some embodiments, the individual is undergoing a coronary stent procedure.

In some embodiments, the individual is undergoing angioplasty with stent.

In some embodiments, the individual has coronary artery disease (CAD).

In some embodiments, the individual has complex coronary artery disease.

In some embodiments, the individual has acute coronary syndrome.

In some embodiments, the individual has nonobstructive coronary artery disease (NOCA).

In some embodiments, the individual has angina and NOCA.

In some embodiments, the individual has nonobstructive coronary artery disease (NCAD).

In some embodiments, the individual has acute coronary syndrome with nonobstructive coronary artery (ACS-NOCA).

In some embodiments, the individual has troponin-positive non-obstructive coronary arteries (TP-NOCA).

In some embodiments, the individual has myocardial infarction with nonobstructive coronary arteries (MINOCA).

In some embodiments, the individual has peripheral artery disease (PAD).

In some embodiments, the individual has saphenous vein thrombosis.

In some embodiments, the individual has great saphenous vein thrombosis.

In some embodiments, the individual is undergoing a stent graft procedure.

In some embodiments, the microvascular obstruction is saphenous vein graft (SVG) occlusion.

In some embodiments, the individual is undergoing an intervention procedure for coronary artery disease.

In some embodiments, the individual is undergoing an intervention procedure for peripheral artery disease.

In some embodiments, the methods provided herein prevent or reduce platelet aggregation following PCI.

In some embodiments, the methods provided herein are useful for the prevention or treatment of vasoconstriction following PCI.

In some embodiments, the methods provided herein are useful for the prevention or treatment of coronary microvascular obstruction following PCI.

In some embodiments, the methods provided herein are useful for the prevention or treatment of MVO.

In some embodiments, the methods provided herein are useful for the prevention or treatment of MVO in the setting of PCI.

In some embodiments, the methods provided herein are useful for the prevention or treatment of MVO in the setting of PCI for ACS.

In some embodiments, the methods provided herein are useful for the reduction of the incidence of MVO at about 2 to about 30 days post-PCI.

In some embodiments, the methods provided herein are useful for the prevention or treatment of myocardial injury following PCI.

In some embodiments, the methods provided herein are useful for the improvement of cardiac function following PCI.

In some embodiments, the methods provided herein are useful for cardiac recovery following PCI.

In some embodiments, the methods provided herein are useful for the improvement of an LV function parameter. In some embodiments, the methods provided herein are useful for the improvement of LV ejection fraction (LVEF). In some embodiments, the methods provided herein are useful for the improvement of LV end-diastolic volume (LVEDV). In some embodiments, the methods provided herein are useful for the improvement of LV end-systolic volume (LVESV).

In some embodiments, the methods provided herein are useful for the improvement of post-PCI cardiac recovery.

In some embodiments, the methods provided herein are useful for the improvement of post-PCI clinical outcomes.

In some embodiments, the methods provided herein are useful for the improvement of myocardial perfusion following PCI.

In some embodiments, the methods provided herein are useful for the prevention or treatment of damaged vasculature following PCI.

In some embodiments, the methods provided herein are useful for the reduction of adverse effects of microcirculation as endothelium recovers following PCI.

In some embodiments, the methods described herein are useful for the reduction of the incidence of MACE. In some embodiments, MACE is the composite of cardiac death, myocardial re-infarction, clinically driven myocardial revascularization, and hospitalization with discharge diagnosis of heart failure or coronary artery disease, during the 90 days (or 3 months) post-PCI. In some embodiments, the methods described herein are for the prevention or reduction of risk of myocardial infarction, heart failure, and/or death.

In some embodiments, the ACS is ST-elevation myocardial infarction (STEMI).

In some embodiments, the parenteral formulation of Compound 1, or a pharmaceutically acceptable salt, hydrate, or solvate thereof, is administered immediately prior to PCI. In some embodiments, the parenteral formulation is administered within, or within about, 5, 10, 15, 20, 25, 30, 35, 40, 45, 50, 55, or 60 minutes of PCI. In some embodiments, the parenteral formulation of Compound 1, or a pharmaceutically acceptable salt, hydrate, or solvate thereof, is administered during PCI.

In some embodiments, the parenteral formulation is administered by infusion.

In some embodiments, the parenteral administration comprises administration of an injection and an infusion.

In some embodiments, the oral administration comprises an oral loading dose and an oral maintenance dose.

In some embodiments, about 5, 10, 15, 20, 25, 30, 35, 40, 45, 50, 55, 60, 65, 70, or 75 mg of the Compound 1, or a pharmaceutically acceptable salt, hydrate, or solvate thereof is administered by injection

In some embodiments, about 5, 10, 15, 20, 25, 30, 35, 40, 45, 50, 55, 60, 65, 70, or 75 mg of the Compound 1, or a pharmaceutically acceptable salt, hydrate, or solvate thereof is administered by infusion

In some embodiments, the parenteral formulation is administered over about 5 minutes or less, 4 minutes or less, 3 minutes or less, 2 minutes or less, or 1 minute or less.

In some embodiments, about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, or 160 mg of the Compound 1 or pharmaceutically acceptable salt, hydrate, or solvate thereof is administered to the individual through the oral formulation.

In some embodiments, the parenteral formulation is administered once.

In some embodiments, the first period of time is, or is about, less than about, 15, 14, 13, 12, 11, 10, 9, 8, 7, 6, 5, 4, 3, 2, or 1 minute(s). In some embodiments, first period of time is about, or less than about, 2 minutes, 3 minutes, 4 minutes, 5 minutes, 6 minutes, 7 minutes, 8 minutes, 9 minutes, or 10 minutes. In some embodiments, first period of time is about, or less than about, 5 minutes.

In some embodiments, about 20 mg of parenteral formulation is administered over about, or at least about, 2 minutes. In some embodiments, about 30 mg of parenteral formulation is administered over about, or at least about, 5 minutes. In some embodiments, about 40 mg of parenteral formulation is administered over about, or at least about, 5 minutes. In some embodiments, about, or less than about, 5, 6, 7, 8, 9, 10, 11, 12, 13, 14, or 15 mg of a parenteral formulation is administered per minute.

In some embodiments, the parenteral formulation is administered as a single dose. In some embodiments, the parenteral formulation is administered as a bolus. In some embodiments, the parenteral formulation is administered as a single bolus. In some embodiments, the parenteral formulation is administered as a single IV bolus. In some embodiments, the parenteral formulation is administered with an infusion pump. In some embodiments, the parenteral formulation is administered as an IV injection. In some embodiments, the parenteral formulation is administered as a single IV injection. In some embodiments, a combination of parenteral formulations is administered to maintain a target plasma concentration, such as a target Cmin. For example, in some embodiments, the parenteral formulation is administered as a bolus followed by an infusion. In some embodiments, the parenteral formulation is administered as an IV bolus followed by an IV infusion. In some embodiments, the parenteral formulation is administered as an infusion followed by a bolus. In some embodiments, the parenteral formulation is administered as an IV infusion followed by an IV bolus. In some embodiments, the parenteral formulation is administered following a loading dose. In some embodiments, the parenteral formulation or parenteral formulations are administered following an oral loading dose. In some embodiments, the oral loading dose is followed by an oral maintenance dose.

In some embodiments, the Compound 1, or a pharmaceutically acceptable salt, hydrate, or solvate thereof, is administered as an IV bolus, followed by an IV infusion, followed by an oral dosage form. In some embodiments, the IV infusion is started at, or at about, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, or 6 hours following administration of the IV bolus. In some embodiments, the oral dosage form is administered at, or at about, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, or 8 hours following administration of the IV bolus. In some embodiments, the oral dosage form is administered at, or at about, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, or 8 hours following the administration of the IV infusion. In some embodiments, an IV bolus is administered, followed by an IV infusion about one hour following the start of the IV bolus, followed by an oral administration about 5 hours following the start of the IV bolus.

In some embodiments, the Compound 1, or a pharmaceutically acceptable salt, hydrate, or solvate thereof, is administered as an IV infusion, followed by an IV bolus, followed by an oral dosage form. In some embodiments, the Compound 1, or a pharmaceutically acceptable salt, hydrate, or solvate thereof, is administered as an IV infusion, followed by an IV bolus, followed by an oral loading dose, followed by an oral maintenance dose. In some embodiments, the Compound 1, or a pharmaceutically acceptable salt, hydrate, or solvate thereof, is administered as an IV infusion, followed by an oral loading dose, followed by an oral maintenance dose. In some embodiments, the Compound 1, or a pharmaceutically acceptable salt, hydrate, or solvate thereof, is administered as an IV bolus, followed by an oral loading dose, followed by an oral maintenance dose.

In some embodiments, the parenteral formulation is administered over about, or less than about, 10, 9, 8, 7, 6, 5, 4, 3, 2, or 1 minute(s). In some embodiments, the parenteral formulation is administered over about, or less than about, 5 minutes. In some embodiments, the parenteral formulation is administered as a bolus at a rate that does not exceed 3, 2.75, 2.5, 2.25, or 2 mL of Compound 1, or a pharmaceutically acceptable salt, hydrate, or solvate thereof, per minute.

In some embodiments, the oral formulation of Compound 1, or a pharmaceutically acceptable salt, hydrate, or solvate thereof, is administered following PCI. In some embodiments, the first oral formulation of Compound 1, or a pharmaceutically acceptable salt, hydrate, or solvate thereof, is administered the day of the PCI procedure. In some embodiments, the first oral formulation of Compound 1, or a pharmaceutically acceptable salt, hydrate, or solvate thereof, is administered about 1, 2, 3, 4, 5, 6, 7, 8, 9, or hours following the PCI procedure. In some embodiments, the first oral formulation of Compound 1, or a pharmaceutically acceptable salt, hydrate, or solvate thereof, is administered about 4 hours following the PCI procedure. In some embodiments, the first oral formulation of Compound 1, or a pharmaceutically acceptable salt, hydrate, or solvate thereof, is administered about 1, 2, 3, 4, 5, 6, 7, 8, 9, or 10 hours following administration of the parenteral dose. In some embodiments, the first oral formulation of Compound 1, or a pharmaceutically acceptable salt, hydrate, or solvate thereof, is administered about 4 hours following administration of the parenteral dose. In some embodiments, the first oral formulation of Compound 1, or a pharmaceutically acceptable salt, hydrate, or solvate thereof, is administered the day after the PCI procedure. In some embodiments, the first oral formulation of Compound 1, or a pharmaceutically acceptable salt, hydrate, or solvate thereof, is administered within a week of the PCI procedure.

In some embodiments, the target plasma concentration following administration of a parenteral formulation is, or is about, 5-200 ng/mL. In some embodiments, the target plasma concentration following administration of the parenteral formulation is, or is about, 60-100 ng/mL. In some embodiments, the target plasma concentration following administration of the parenteral formulation is, or is about, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 ng/mL. In some embodiments, the target plasma concentration is C min. In some embodiments, the target plasma concentration is a measurement of Compound 1 and its metabolites. In some embodiments, the target plasma concentration is a measurement of Compound 1, M1, and M2.

In some embodiments, the oral formulation is administered once daily (QD). In some embodiments, the oral formulation is administered twice daily (BID). In some embodiments, the oral formulation is administered three times daily (TID).

In some embodiments, the second period of time is at least, or at least about, 1, 2, 3, 4, 5, 6, 7, 8, 9, 10, 11, or 12 months. In some embodiments, the second period of time is at least one week. In some embodiments, the second period of time is at least one month (or 30 days). In some embodiments, the second period of time is at least three months (or 90 days). In some embodiments, the second period of time is at least six months (or 180 days). In some embodiments, the second period of time is at least one year.

In some embodiments, the incidence of MVO is measured using cardiac magnetic resonance (CMR) imaging.

In some embodiments, the incidence of MVO is measured using angiographic assessment of the myocardial blush grade (MBG).

In some embodiments, the incidence of MVO is measured using an assessment of coronary physiology, based on an index of microcirculatory resistance (IMR).

In some embodiments, the Compound 1, or a pharmaceutically acceptable salt, hydrate, or solvate thereof, is a pharmaceutically acceptable salt of Compound 1, or a hydrate or solvate thereof.

In some embodiments, the Compound 1, or a pharmaceutically acceptable salt, hydrate, or solvate thereof, is a pharmaceutically acceptable salt of Compound 1.

In some embodiments, the Compound 1, or a pharmaceutically acceptable salt, hydrate, or solvate thereof, is a HCl salt of Compound 1. In some embodiments, the Compound 1, or a pharmaceutically acceptable salt, hydrate, or solvate thereof, is a hydrate of Compound 1. In some embodiments, the Compound 1, or a pharmaceutically acceptable salt, hydrate, or solvate thereof, is a solvate of Compound 1.

### Parenteral Administration

Parenteral dosage forms can be prepared by dissolving the compound in a suitable liquid vehicle and filter sterilizing the solution before filling and sealing an appropriate vial or ampule. These are just a few examples of the many appropriate methods well known in the art for preparing dosage forms. Suitable pharmaceutically-acceptable carriers, outside those mentioned herein, are known in the art. See. e.g., Remington. The Science and Practice of Pharmacy, 20^{th} Edition, 2000. Lippincott Williams & Wilkins. (Editors: Gennaro et al.).

Accordingly, also provided is a pharmaceutical formulation for parenteral administration comprising a sterilized solution. In some embodiments, the solution comprises, or comprises about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/mL (adjusted free-base concentration) of Compound 1, or a pharmaceutically acceptable salt, hydrate, or solvate thereof. In some embodiments, the solution comprises, or comprises about, 4 mg/mL (adjusted free-base concentration) of Compound 1, or a pharmaceutically acceptable salt, hydrate, or solvate thereof. In some embodiments, the solution comprises, or comprises about, 8 mg/mL (adjusted free-base concentration) of Compound 1, or a pharmaceutically acceptable salt, hydrate, or solvate thereof. In some embodiments, the solution comprises, or comprises about, 25 mg/mL (adjusted free-base concentration) of Compound 1, or a pharmaceutically acceptable salt, hydrate, or solvate thereof. In some embodiments, the solution is diluted prior to administration. In some embodiments, the solution is diluted with saline prior to administration. In some embodiments, the solution as administered comprises, or comprises about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/mL (adjusted free-base concentration) of Compound 1, or a pharmaceutically acceptable salt, hydrate, or solvate thereof. In some embodiments, the solution as administered comprises less than, or less than about, 25 mg/mL (adjusted free-base concentration) of Compound 1, or a pharmaceutically acceptable salt, hydrate, or solvate thereof. In some embodiments, the solution as administered comprises less than, or less than about, 25, 24, 23, 22, 21, 20, 19, 18, 17, 16, 15, 14, 13, 12, 11, 10, 9, 8, 7, 6, or 5 mg/mL (adjusted free-base concentration) of Compound 1, or a pharmaceutically acceptable salt, hydrate, or solvate thereof.

The formulation typically includes a buffer. Suitable buffers that can be used in the compositions of the invention include, e.g., histidine buffer, acetate buffer, citrate buffer, tartrate buffer, lactate buffer, succinate buffer and phosphate buffer. The pH of the buffer will typically be between about 2 to about 10, e.g., about pH 2, 3, 4, 5, 6, 7, 8, 9 or 10. The pH of the buffer will preferably be about 4 to about 8. In some embodiments, the pH of the solution is buffered at, or at about, a pH of 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5. In some embodiments, the pH of the solution is buffered at, or at about, a pH of 3 to 4. In some embodiments, the pH of the solution is buffered at, or at about, a pH of 3.5. In some embodiments, a citrate buffer, such as a 10 mM citrate buffer, is used. In some embodiments, an acetate buffer is used. In some embodiments, the aqueous buffer is present in the formulation at a concentration of, or of about, 0.5 to 5 mg/mL per 25 mg/mL of adjusted free-base concentration of Compound 1. In some embodiments, the aqueous buffer is present in the formulation at a concentration of, or of about, 0.5, 0.75, 1, 1.25, 1.5, 1.75, 2, 2.25, 2.75, 3, 3.25, 3.5, 3.75, 4, 4.25, 4.5, 4.75 or 5 mg/mL per 25 mg/mL of adjusted free-base concentration of Compound 1.

In some embodiments, the pharmaceutical formulation for parenteral administration comprises:
an aqueous, 10 mM citrate buffer;
3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, at a concentration of 25 mg/mL (adjusted free-base concentration);
and a tonicity adjuster.

In some embodiments, the tonicity adjuster is an ionic or non-ionic tonicity adjuster, e.g., glycerin, sugars (including glucose, mannitol, sorbitol, trehalose, dextrose, lactose, etc.), sodium chloride or sodium sulphate. In some embodiments, the tonicity adjuster comprises dextrose. In some embodiments, the tonicity adjuster comprises saline.

In some embodiments, the tonicity adjuster is present in the formulation in an amount from about 0.1% to about 30% by weight of the compositions, e.g., about 0.1%, 0.2%, 0.3%, 0.4%, 0.5%, 0.6%, 0.7%, 0.8%, 0.9%, 1%, 2.5%, 5%, 10%, 15%, 20%, 25% or 30% by weight of the formulation. The formulations may be hypotonic, isotonic or hypertonic and typically have an osmolality of from about 200 to about 350 mOsmol/kg. For example, the compositions may have an osmolality of, or of about, 200, 210, 220, 230, 240, 250, 260, 270, 280, 290, 300, 310, 320, 330, 340 or 350 mOsmol/kg. In some embodiments, the compositions have an osmolality of, or of about, 300 mOsmol/kg. The skilled person will understand that the amount of tonicity adjuster used may vary depending on the particular choice of tonicity adjuster and on the other components in the composition. In some embodiments, the tonicity adjuster is at a concentration of, or of about, 200, 225, 250, 275, 200, 325, or 350 mM. In some embodiments, the tonicity adjuster is at a concentration of, or of about, 289 mM. In some embodiments, the parenteral formulation is isotonic. In some embodiments, the tonicity adjuster is present in the formulation at a concentration of, or of about, 10 to 50 mg/mL per 25 mg/mL of adjusted free-base concentration of Compound 1. In some embodiments, the tonicity adjuster is present in the formulation at a concentration of, or of about, 10, 15, 20, 25, 30, 35, 40, 45, or 50 mg/mL per 25 mg/mL of adjusted free-base concentration of Compound 1.

In some embodiments, Compound 1, or a pharmaceutically acceptable salt, hydrate, or solvate thereof, is administered parenterally in an amount equivalent to between about 10 mg and about 100 mg of Compound 1 free base.

In some embodiments, Compound 1, or a pharmaceutically acceptable salt, hydrate, or solvate thereof, is administered parenterally in an amount equivalent to between about 10 mg and about 75 mg of Compound 1 free base.

In some embodiments, Compound 1, or a pharmaceutically acceptable salt, hydrate, or solvate thereof, is administered parenterally in an amount equivalent to between about 10 mg and about 40 mg of Compound 1 free base.

In some embodiments, Compound 1, or a pharmaceutically acceptable salt, hydrate, or solvate thereof, is administered parenterally in an amount equivalent to about 10 mg of Compound 1 free base. In some embodiments, Compound 1, or a pharmaceutically acceptable salt, hydrate, or solvate thereof, is administered parenterally in an amount equivalent to about 15 mg of Compound 1 free base. In some embodiments, Compound 1, or a pharmaceutically acceptable salt, hydrate, or solvate thereof, is administered parenterally in an amount equivalent to about 20 mg of Compound 1 free base. In some embodiments, Compound 1, or a pharmaceutically acceptable salt, hydrate, or solvate thereof, is administered parenterally in an amount equivalent to about 25 mg of Compound 1 free base. In some embodiments, the Compound 1, or a pharmaceutically acceptable salt, hydrate, or solvate thereof, is administered parenterally in an amount equivalent to about 30 mg of Compound 1 free base. In some embodiments, Compound 1, or a pharmaceutically acceptable salt, hydrate, or solvate thereof, is administered parenterally in an amount equivalent to about 35 mg of Compound 1 free base. In some embodiments, Compound 1, or a pharmaceutically acceptable salt, hydrate, or solvate thereof, is administered parenterally in an amount equivalent to about 40 mg of Compound 1 free base. In some embodiments, Compound 1, or a pharmaceutically acceptable salt, hydrate, or solvate thereof, is administered parenterally in an amount equivalent to about 45 mg of Compound 1 free base. In some embodiments, the Compound 1, or a pharmaceutically acceptable salt, hydrate, or solvate thereof, is administered parenterally in an amount equivalent to about 50 mg of Compound 1 free base. In some embodiments, Compound 1, or a pharmaceutically acceptable salt, hydrate, or solvate thereof, is administered parenterally in an amount equivalent to about 55 mg of Compound 1 free base. In some embodiments, Compound 1, or a pharmaceutically acceptable salt, hydrate, or solvate thereof, is administered parenterally in an amount equivalent to about 60 mg of Compound 1 free base. In some embodiments, Compound 1, or a pharmaceutically acceptable salt, hydrate, or solvate thereof, is administered parenterally in an amount equivalent to about 65 mg of Compound 1 free base. In some embodiments, the Compound 1, or a pharmaceutically acceptable salt, hydrate, or solvate thereof, is administered parenterally in an amount equivalent to about 70 mg of Compound 1 free base. In some embodiments, Compound 1, or a pharmaceutically acceptable salt, hydrate, or solvate thereof, is administered parenterally in an amount equivalent to about 75 mg of Compound 1 free base. In some embodiments, Compound 1, or a pharmaceutically acceptable salt, hydrate, or solvate thereof, is administered parenterally in an amount equivalent to about 80 mg of Compound 1 free base. In some embodiments, Compound 1, or a pharmaceutically acceptable salt, hydrate, or solvate thereof, is administered parenterally in an amount equivalent to about 85 mg of Compound 1 free base. In some embodiments, the Compound 1, or a pharmaceutically acceptable salt, hydrate, or solvate thereof, is administered parenterally in an amount equivalent to about 90 mg of Compound 1 free base. In some embodiments, Compound 1, or a pharmaceutically acceptable salt, hydrate, or solvate thereof, is administered parenterally in an amount equivalent to about 95 mg of Compound 1 free base. In some embodiments, the Compound 1, or a pharmaceutically acceptable salt, hydrate, or solvate thereof, is administered parenterally in an amount equivalent to about 100 mg of Compound 1 free base.

In some embodiments, the total daily dose of parenterally administered Compound 1, or a pharmaceutically acceptable salt, hydrate, or solvate thereof, is, or is about, 10 to 100 mg. In some embodiments, the total daily dose of parenterally administered Compound 1, or a pharmaceutically acceptable salt, hydrate, or solvate thereof, is, or is about, 10, 15, 20, 25, 30, 35, 40, 45, 50, 55, 60, 65, 70, 75, 80, 85, 90, 95, or 100 mg.

In some embodiments, the total daily dose of parenterally and orally administered Compound 1, or a pharmaceutically acceptable salt, hydrate, or solvate thereof, is, or is about, 200 to 500 mg. In some embodiments, the total daily dose of parenterally and orally administered Compound 1, or a pharmaceutically acceptable salt, hydrate, or solvate thereof, is, or is about, 200, 220, 225, 240, 250, 260, 275, 280, 300, 320, 325, 340, 350, 360, 375, 380, 400, 420, 425, 440, 450, 460, 475, 480, or 500 mg.

In some embodiments, about 10-20 mg of Compound 1, or a pharmaceutically acceptable salt, hydrate, or solvate thereof, is administered by IV; about 40 mg of Compound 1, or a pharmaceutically acceptable salt, hydrate, or solvate thereof, is administered by infusion; and about 100-150 mg of Compound 1, or a pharmaceutically acceptable salt, hydrate, or solvate thereof, is administered orally TID.

In some embodiments, about 10-20 mg of Compound 1, or a pharmaceutically acceptable salt, hydrate, or solvate thereof, is administered by IV; about 40 mg of Compound 1, or a pharmaceutically acceptable salt, hydrate, or solvate thereof, is administered by infusion; about 200 mg of Compound 1, or a pharmaceutically acceptable salt, hydrate, or solvate thereof is administered as an oral loading dose; and about 100-150 mg of Compound 1, or a pharmaceutically acceptable salt, hydrate, or solvate thereof, is administered orally TID as a maintenance dose.

### Oral Administration

In some embodiments, the Compound 1, or a pharmaceutically acceptable salt, hydrate, or solvate thereof, is administered orally.

The compounds provided herein, together with a conventional adjuvant, carrier, or diluent, can thus be placed into the form of pharmaceutical formulations and unit dosages thereof and in such form may be employed as solids, such as tablets or filled capsules, or liquids, such as solutions, suspensions, emulsions, elixirs, gels or capsules filled with the same, all for oral use. Such pharmaceutical compositions and unit dosage forms thereof can comprise conventional ingredients in conventional proportions, with or without additional active compounds or principles and such unit dosage forms may contain any suitable effective amount of the active ingredient commensurate with the intended daily dosage range to be employed. In some embodiments, the capsule is a gelatin capsule. In some embodiments, the capsule is a hard gelatin capsule. In some embodiments, the oral formulation is powder in a capsule.

For oral administration, the pharmaceutical composition can be in the form of, for example, a tablet, capsule, suspension or liquid. The pharmaceutical composition is made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate.

In some embodiments, the oral formulation of Compound 1 or pharmaceutically acceptable salt, hydrate, or solvate thereof, is an immediate-release dosage form comprising Compound 1, or a pharmaceutically acceptable salt, hydrate, or solvate thereof.

In some embodiments, the Compound 1, or a pharmaceutically acceptable salt, hydrate, or solvate thereof, is formulated as a capsule suitable for oral administration. In some embodiments, the Compound 1, or a pharmaceutically acceptable salt, hydrate, or solvate thereof, is formulated as a tablet suitable for oral administration.

In some embodiments, Compound 1, or a pharmaceutically acceptable salt, hydrate, or solvate thereof, is orally administered in an amount equivalent to between about 10 mg and about 250 mg of Compound 1 free base.

In some embodiments, Compound 1, or a pharmaceutically acceptable salt, hydrate, or solvate thereof, is administered orally in an amount equivalent to between about 20 mg and about 250 mg of Compound 1 free base.

In some embodiments, Compound 1, or a pharmaceutically acceptable salt, hydrate, or solvate thereof, is administered orally in an amount equivalent to between about 10 mg and about 200 mg of Compound 1 free base.

In some embodiments, Compound 1, or a pharmaceutically acceptable salt, hydrate, or solvate thereof, is administered orally in an amount equivalent to between about 10 mg and about 80 mg of Compound 1 free base.

In some embodiments, Compound 1, or a pharmaceutically acceptable salt, hydrate, or solvate thereof, is administered orally in an amount equivalent to between about 10 mg and about 60 mg of Compound 1 free base.

In some embodiments, Compound 1, or a pharmaceutically acceptable salt, hydrate, or solvate thereof, is administered orally in an amount equivalent to about 10 mg of Compound 1 free base. In some embodiments, Compound 1, or a pharmaceutically acceptable salt, hydrate, or solvate thereof, is administered orally in an amount equivalent to about 15 mg of Compound 1 free base. In some embodiments, Compound 1, or a pharmaceutically acceptable salt, hydrate, or solvate thereof, is administered in an amount equivalent to about 20 mg of Compound 1 free base. In some embodiments, Compound 1, or a pharmaceutically acceptable salt, hydrate, or solvate thereof, is orally administered in an amount equivalent to about 25 mg of Compound 1 free base. In some embodiments, the Compound 1, or a pharmaceutically acceptable salt, hydrate, or solvate thereof, is orally administered in an amount equivalent to about 30 mg of Compound 1 free base. In some embodiments, Compound 1, or a pharmaceutically acceptable salt, hydrate, or solvate thereof, is orally administered in an amount equivalent to about 40 mg of Compound 1 free base. In some embodiments, the Compound 1, or a pharmaceutically acceptable salt, hydrate, or solvate thereof, is orally administered in an amount equivalent to about 50 mg of Compound 1 free base. In some embodiments, Compound 1, or a pharmaceutically acceptable salt, hydrate, or solvate thereof, is orally administered in an amount equivalent to about 60 mg of Compound 1 free base. In some embodiments, the Compound 1, or a pharmaceutically acceptable salt, hydrate, or solvate thereof, is orally administered in an amount equivalent to about 70 mg of Compound 1 free base. In some embodiments, Compound 1, or a pharmaceutically acceptable salt, hydrate, or solvate thereof, is orally administered in an amount equivalent to about 75 mg of Compound 1 free base. In some embodiments, Compound 1, or a pharmaceutically acceptable salt, hydrate, or solvate thereof, is orally administered in an amount equivalent to about 80 mg of Compound 1 free base. In some embodiments, the Compound 1, or a pharmaceutically acceptable salt, hydrate, or solvate thereof, is orally administered in an amount equivalent to about 90 mg of Compound 1 free base. In some embodiments, the Compound 1, or a pharmaceutically acceptable salt, hydrate, or solvate thereof, is orally administered in an amount equivalent to about 100 mg of Compound 1 free base. In some embodiments, the Compound 1, or a pharmaceutically acceptable salt, hydrate, or solvate thereof, is orally administered in an amount equivalent to about 110 mg of Compound 1 free base.

In some embodiments, the Compound 1, or a pharmaceutically acceptable salt, hydrate, or solvate thereof, is orally administered in an amount equivalent to about 120 mg of Compound 1 free base. In some embodiments, the Compound 1, or a pharmaceutically acceptable salt, hydrate, or solvate thereof, is orally administered in an amount equivalent to about 130 mg of Compound 1 free base. In some embodiments, the Compound 1, or a pharmaceutically acceptable salt, hydrate, or solvate thereof, is orally administered in an amount equivalent to about 140 mg of Compound 1 free base. In some embodiments, the Compound 1, or a pharmaceutically acceptable salt, hydrate, or solvate thereof, is orally administered in an amount equivalent to about 150 mg of Compound 1 free base. In some embodiments, the Compound 1, or a pharmaceutically acceptable salt, hydrate, or solvate thereof, is orally administered in an amount equivalent to about 160 mg of Compound 1 free base. In some embodiments, the Compound 1, or a pharmaceutically acceptable salt, hydrate, or solvate thereof, is orally administered in an amount equivalent to about 170 mg of Compound 1 free base. In some embodiments, the Compound 1, or a pharmaceutically acceptable salt, hydrate, or solvate thereof, is orally administered in an amount equivalent to about 180 mg of Compound 1 free base. In some embodiments, the Compound 1, or a pharmaceutically acceptable salt, hydrate, or solvate thereof, is orally administered in an amount equivalent to about 190 mg of Compound 1 free base.

In some embodiments, the Compound 1, or a pharmaceutically acceptable salt, hydrate, or solvate thereof, is orally administered in an amount equivalent to about 200 mg of Compound 1 free base. In some embodiments, the Compound 1, or a pharmaceutically acceptable salt, hydrate, or solvate thereof, is orally administered in an amount equivalent to about 210 mg of Compound 1 free base. In some embodiments, the Compound 1, or a pharmaceutically acceptable salt, hydrate, or solvate thereof, is orally administered in an amount equivalent to about 220 mg of Compound 1 free base. In some embodiments, the Compound 1, or a pharmaceutically acceptable salt, hydrate, or solvate thereof, is orally administered in an amount equivalent to about 230 mg of Compound 1 free base. In some embodiments, the Compound 1, or a pharmaceutically acceptable salt, hydrate, or solvate thereof, is orally administered in an amount equivalent to about 240 mg of Compound 1 free base. In some embodiments, the Compound 1, or a pharmaceutically acceptable salt, hydrate, or solvate thereof, is orally administered in an amount equivalent to about 250 mg of Compound 1 free base.

In some embodiments, an oral loading dose of, or of about, 75, 80, 85, 90, 95, 100, 105, 110, 120, 125, 130, 135, 140, 145, 150, 155, 160, 165, 170, 175, 180, 185, 190, 195, 200, 205, 210, 215, 220, 225, 230, 2356, 240, 245, or 250 mg of Compound 1, or a pharmaceutically acceptable salt, hydrate, or solvate thereof, is administered to the individual.

In some embodiments, the Compound 1, or a pharmaceutically acceptable salt, hydrate, or solvate thereof, is orally administered once daily (QD). In some embodiments, the QD dose is a maintenance dose.

In some embodiments, the Compound 1, or a pharmaceutically acceptable salt, hydrate, or solvate thereof, is orally administered twice daily (BID). In some embodiments, the BID dose is a maintenance dose.

In some embodiments, the Compound 1, or a pharmaceutically acceptable salt, hydrate, or solvate thereof, is orally administered three times daily (TID). In some embodiments, the TID dose is a maintenance dose.

In some embodiments, Compound 1, or a pharmaceutically acceptable salt, hydrate, or solvate thereof, is orally administered in an amount equivalent to between about 10 mg and about 80 mg of Compound 1 free base TID (for a total of between about 30 mg and about 240 mg of Compound 1 free base daily).

In some embodiments, Compound 1, or a pharmaceutically acceptable salt, hydrate, or solvate thereof, is orally administered in an amount equivalent to between about 10 mg and about 60 mg of Compound 1 free base TID (for a total of between about 30 mg and about 180 mg of Compound 1 free base daily).

In some embodiments, the oral formulation is orally administered for at least, or at least about, 1, 2, 3, 4, 5, 6, 7, 8, 9, 10, 11, or 12 months.

In some embodiments, an oral maintenance dose of, or of about, 75, 80, 85, 90, 95, 100, 105, 110, 120, 125, 130, 135, 140, 145, or 150 mg of Compound 1, or a pharmaceutically acceptable salt, hydrate, or solvate thereof, is administered to the individual. In some embodiments, the oral maintenance dose is administered TID.

In some embodiments, the total daily dose of orally administered Compound 1, or a pharmaceutically acceptable salt, hydrate, or solvate thereof, is, or is about, 100 to 400 mg. In some embodiments, the total daily dose of orally administered Compound 1, or a pharmaceutically acceptable salt, hydrate, or solvate thereof, is, or is about, 100, 120, 125, 140, 150, 160, 175, 180, 200, 220, 225, 240, 250, 260, 275, 280, 300, 320, 325, 340, 350, 360, 375, 380, or 400 mg.

### Co-Administration

In some embodiments, the Compound 1, or a pharmaceutically acceptable salt, hydrate, or solvate thereof, is co-administered with at least one anti-platelet drug. In some embodiments, the Compound 1, or a pharmaceutically acceptable salt, hydrate, or solvate thereof, is co-administered with dual anti-platelet therapy (DAPT). In some embodiments, the individual is already being administered at least one anti-platelet drug prior to the administration of Compound 1, or a pharmaceutically acceptable salt, hydrate, or solvate thereof as described herein. In some embodiments, the individual is administered a first dose of at least one anti-platelet drug prior to PCI. In some embodiments, the individual is administered a loading dose of at least one anti-platelet drug prior to PCI. In some embodiments, the Compound 1, or a pharmaceutically acceptable salt, hydrate, or solvate thereof, is co-administered with aspirin and an oral P2Y12 inhibitor. In some embodiments, the anti-platelet drug is aspirin. In some embodiments, the anti-platelet drug is a P2Y12 inhibitor. In some embodiments, the P2Y12 inhibitor is an oral P2Y12 inhibitor. In some embodiments, the P2Y12 inhibitor is selected from clopidogrel, ticlopidine, ticagrelor, prasugrel, and cangrelor. In some embodiments, the DAPT includes aspirin. In some embodiments, the DAPT includes clopidogrel. In some embodiments, the DAPT is aspirin and clopidogrel. In some embodiments, the Compound 1, or a pharmaceutically acceptable salt, hydrate, or solvate thereof, is co-administered with clopidogrel, but not prasugrel or ticagrelor. In some embodiments, the Compound 1, or a pharmaceutically acceptable salt, hydrate, or solvate thereof, is co-administered with heparin.

In some embodiments, an individual is not co-administered an inhibitor or inducer of CYP3A4, CYP3A5, and/or P-glycoprotein with Compound 1, or a pharmaceutically acceptable salt, hydrate, or solvate thereof. In some embodiments, the individual is already being administered at least one inhibitor or inducer of CYP3A4, CYP3A5, and/or P-glycoprotein prior to the administration of Compound 1, or a pharmaceutically acceptable salt, hydrate, or solvate thereof as described herein. In some embodiments, the administration of an inhibitor or inducer of CYP3A4, CYP3A5, and/or P-glycoprotein is discontinued when an individual is administered Compound 1, or a pharmaceutically acceptable salt, hydrate, or solvate thereof. In some embodiments, an individual is co-administered a lower dose of an inhibitor or inducer of CYP3A4, CYP3A5, and/or P-glycoprotein when co-administered with Compound 1, or a pharmaceutically acceptable salt, hydrate, or solvate thereof. In some embodiments, an individual is administered a lower dose of Compound 1, or a pharmaceutically acceptable salt, hydrate, or solvate thereof when co-administered with an inhibitor or inducer of CYP3A4, CYP3A5, and/or P-glycoprotein. In some embodiments, an individual is co-administered a lower dose of an inhibitor or inducer of CYP3A4, CYP3A5, and/or P-glycoprotein and a lower dose of Compound 1, or a pharmaceutically acceptable salt, hydrate, or solvate thereof. In some embodiments, the inhibitor or inducer of CYP3A4, CYP3A5, and/or P-glycoprotein is a strong inhibitor or inducer of CYP3A4, CYP3A5, and/or P-glycoprotein. In some embodiments, the inhibitor or inducer of CYP3A4, CYP3A5, and/or P-glycoprotein is selected from ketoconazole, itraconazole, clarithromycin, erythromycin, rifampicin, and verapamil. In some embodiments, the inhibitor or inducer of CYP3A4, CYP3A5, and/or P-glycoprotein is an inhibitor or inducer of CYP3A4. In some embodiments, the inhibitor or inducer of CYP3A4, CYP3A5, and/or P-glycoprotein is an inhibitor or inducer of CYP3A5. In some embodiments, the inhibitor or inducer of CYP3A4, CYP3A5, and/or P-glycoprotein is an inhibitor or inducer of P-glycoprotein. In some embodiments, the individual is co-administered intra-arterial verapamil for radial artery PCI.

Lists of inhibitors, inducers, and substrates can be found, for example, at https://www.fda.gov/drugs/dnig-interactions-labeling/dnig-development-and-dnig-interactions-table-substrates-inhibitors-and-inducers, and other sites.

In some embodiments, the wherein the inhibitor or inducer of CYP3A4, CYP3A5 and/or P-glycoprotein is a CYP3A4/5 inhibitor. In some embodiments, the inhibitor or inducer of CYP3A4, CYP3A5 and/or P-glycoprotein is itraconazole, fluconazole, and/or rifampin.

In some embodiments, the reduced or lower dose of the Compound 1, or a pharmaceutically acceptable salt, hydrate, or solvate thereof, is, or is about, 75%, 70%, 65%, 60%, 55%, 50%, 45%, 400%, 35%, 30%, or 25% of the normal daily dose of the Compound 1, or a pharmaceutically acceptable salt, hydrate, or solvate thereof.

In some embodiments, the CYP3A4/5 inhibitor is itraconazole, ketoconazole, azamulin, troleandomycin, or verapamil. In some embodiments, the CYP3A4/5 inducer is rifampicin. In some embodiments, the CYP3A4/5 substrate is midazolam or triazolam. In some embodiments, the strong inhibitor or CYP3A4 is boceprevir, cobicistat, danoprevir, ritonavir, elvitegravir, grapefruit juice, indinavir, itraconazole, ketoconazole, lopinavir, paritaprevir, ombitasvir, dasabuvir, posaconazole, saquinavir, telaprevir, tipranavir, telithromycin, troleandomycin, and/or voriconazole. In some embodiments, the strong inhibitor or CYP3A4 is clarithromycin, idelalisib, nefazodone, or nelfinavir. In some embodiments, the moderate inhibitor of CYP3A4 is aprepitant, ciprofloxacin, conivaptan, crizotinib, cyclosporine, diltiazem, dronedarone, erythromycm, fluconazole, fluvoxamine, imatinib, tofisopam, or verapamil. In some embodiments, the weak inhibitor of CYP3A4 is chlorzoxazone, cilostazol, cimetidine, clotrimazole, fosaprepitant, istradefylline, ivacaftor, lomitapide, ranitidine, ranolazine, or ticagrelor. In some embodiments, the CYP3A4/5 inhibitor is ketoconazole. In some embodiments, the CYP3A4/5 inhibitor is itraconazole. In some embodiments, the CYP3A4/5 inhibitor is fluconazole.

In some embodiments, the P-glycoprotein substrate is digoxin, fexofenadine, loperamide, quinidine, talinolol, or vinblastine. In some embodiments, the P-glycoprotein inhibitor is cyclosporine, elacidar (GF120918), ketoconazole, quinidine, reserpine, ritonavir, tacrolimus, valspodar (PSC833), verapamil, or zosuquidar (LY335979). In some embodiments, the P-glycoprotein substrate is dabigatran etexilate, digoxin, or fexofenadine. In some embodiments, the P-glycoprotein inhibitor is amiodarone, carvedilol, clarithromycin, dronedarone, itraconazole, lapatinib, lopinavir, ritonavir, propafenone, quinidine, ranolazine, saquinavir, telaprevir, tipranavir, and/or verapamil. In some embodiments, the P-glycoprotein inhibitor is itraconazole. In some embodiments, the proton pump inhibitor is esomeprazole, rlaprazole, lansoprazole, omeprazole, pantoprazole, pantoprazole sodium, pantoprazole magnesium, rabeprazole, or dexlansoprazole. In some embodiments, the proton pump inhibitor is esomeprazole.

Some embodiments include a method of producing a pharmaceutical composition for fixed dose combination therapy comprising admixing at least one compound according to any of the compound embodiments disclosed herein, together with at least one known pharmaceutical agent as described herein and a pharmaceutically acceptable carrier.

Further embodiments include the embodiments disclosed in the following Examples, which are not to be construed as limiting in any way.

### EXAMPLES

### Example 1—Clinical Trial 1

A phase 1, randomized, double-blind, placebo-controlled, single-dose escalation study was conducted to assess the tolerability, PK, and PD of the HCl salt of Compound 1 at doses of 1 to 320 mg (adjusted free-base concentration) administered orally as a single dose to a total of 91 healthy subjects.

Compound 1 was rapidly absorbed at all doses, and plasma exposure measures increased in a greater than dose proportional manner. The active metabolites M1 and M2 had longer half-lives than the parent compound, and as the dose was escalated, Compound 1 half-life increased, exposure increased, and metabolite-to-parent ratios decreased.

PD assessment consisted of measuring pre- and post-dose platelet aggregation using an in vitro assay with ADP with and without 5-HT. The degree of thrombin receptor activating peptide (TRAP)-induced platelet aggregation inhibition between groups was similar. However, Compound 1 inhibited 5-HT-mediated amplification of platelet aggregation at doses above 1 mg.

5-HT-mediated platelet aggregation data from the HCl salt of Compound 1 doses of 5 to 320 mg (adjusted free-base concentration) show a maximal inhibition at 1 to 2 hours and suggest a trend toward longer duration of inhibition with higher doses, although a high degree of variability confounded interpretation.

### Example 2—Clinical Trial 2

A phase 1, randomized, double-blind, placebo-controlled, multiple-ascending dose-escalation study was conducted to assess the safety, tolerability, PD, and steady state PK of the HCl salt of Compound 1 from 5 to 80 mg (adjusted free-base concentration) administered orally TID for 7 days in a total of 50 healthy subjects.

Compound 1 was rapidly absorbed at all doses, and Day 1 AUC and Cmax values at equivalent doses were similar to those for the study in Example 1. Exposure as measured by AUC was dose proportional at 5 and 10 mg, but greater than dose proportional at 40, 60, and 80 mg on Day 1 and Day 7. The Cmax and AUC accumulation ratio values were less than 2 for all doses when comparing Day 1 and Day 7, indicating no accumulation after TID dosing for 7 days. The half-life was similar at all doses and days.

The active metabolite M1 and M2 exposures increased and metabolite to parent ratios decreased with the increasing Compound 1 doses. The accumulation ratios as measured by AUCtau values were approximately 2 at 60 and 80 mg for M1 and M2, suggesting a 2-fold accumulation of these metabolites after 7 days of dosing TID.

Administration of the HCl salt of Compound 1 inhibited ex vivo 5-HT-mediated amplification of platelet aggregation. Inhibition was essentially maximal following dosing on Days 1, 4, and 7 at both the 60 mg and 80 mg doses.

### Example 3—Parenteral Formulation

An example of a parenteral formulation for Compound 1 is provided in Table 1:

TABLE 1
Injectable Solution (25 mg/mL)

| | Concentration | Concentration |
|---|---|---|
| | (mg/mL) | (wt/wt %)^{a} |
| Compound 1 HCl | 27.09^{b} | 2.672 |
| Citric acid, monohydrate | 1.558 | 0.154 |
| Sodium citrate (tribasic), dihydrate | 0.761 | 0.0750 |
| Dextrose, anhydrous | 27.70 | 2.732 |
| Water for Injection | 956.9^{c} | 94.37^{c} |

^{a}Measured density of the formulation is 1.0140 g/mL at 25° C.
^{b}Quantity of Compound 1 HCl salt required to provide 25 mg of Compound 1 free base.
^{c}Calculated from measured density.

### Example 4—Oral Formulation

An example of an oral formulation for Compound 1 is provided in Table 2:

TABLE 2
Immediate-Release Capsule

| | mg/capsule |
|---|---|
| Compound 1 HCl | 21.67 |
| Hard gelatin capsule | 61.00^{d} |
| Total | 82.67^{d} |

^{d}approximate weight based on a typical empty capsule weight.

### Example 5—Clinical Trial 3

A phase 1, randomized, blinded, sponsor-open, placebo-controlled, single ascending dose (SAD) study was conducted to assess the safety, tolerability, PK, and PD of single IV doses of Compound 1 and two active metabolites (M1 and M2) in healthy adult subjects. Five dose cohorts (each cohort composed of six Compound 1 subjects and two placebo subjects) were evaluated.

Subjects received a single dose of Compound 1 or placebo. Each cohort included a sentinel group of two subjects (one active and one placebo) dosed at least 24 hours before the remaining six subjects (five active and one placebo). The remaining subjects were dosed following review of all available safety and tolerability data from the sentinel subjects. Safety, tolerability, and PK data were reviewed after the completion of each dose cohort and prior to escalation to the next dose cohort. PD data were reviewed prior to dosing subsequent cohorts. Selection of dose for subsequent cohorts was based on the data from previous cohort(s). Following administration of Compound 1 or placebo, subjects were confined for 72 hours to monitor safety and to collect PK/PD samples. Cardiodynamic electrocardiogram (ECG) sampling were collected pre-dose and through 24 hours post-dose in each cohort. The clinic contacted subjects on Day 11 (1 day) to determine if any adverse event (AE) occurred since discharge.

IV doses of placebo (n=35), 1 mg Compound 1 (n=6), 5 mg Compound 1 (n=6), 10 mg Compound 1 (n=5), 20 mg Compound 1 (n=6), 30 mg Compound 1 (n=6), or 40 mg Compound 1 (n=6) were administered. The doses of 1 mg, 5 mg, 10 mg, and 20 mg of Compound 1 were administered by slow bolus infusion over a period of two minutes, while the doses of 30 mg and 40 mg of Compound 1 were administered over a period of five minutes. The PD effects of the single doses were examined immediately after dosing and 4 hours after dosing.

No subjects discontinued treatment. The most common treatment-emergent adverse events occurred near the site of venipuncture. Epistaxis occurred in one subject in the Compound 1 group and one subject in the placebo group. Epistaxis occurred in one subject in the Compound 1 group and one subject in the placebo group. None of the bleeding-related TEAEs were greater than a Bleeding Academic Research Consortium (BARC) Type 1.

The mean observed Cmax at the 40 mg dose was 1840 ng/mL. The mean terminal elimination half-life ranged from 1 to 2 hours and was dose-dependent. The mean plasma exposures to active metabolites were less than 15% and 2% of Compound 1 mean plasma exposure. Near-maximal inhibition of 5-HT-mediated amplification of platelet aggregation was observed with doses of 20 mg and greater of Compound 1.

### Example 6—Clinical Trial 4

A phase 2b, multicenter, randomized, double-blind, placebo-controlled study will be conducted to assess the safety, tolerability, and effects of Compound 1, or a pharmaceutically acceptable salt thereof, on the prevention and treatment of MVO in subjects who are undergoing PCI for ACS (STEMI, or NSTEMI within 24 hours of diagnosis). Prior to performing the PCI procedure on Day 1, subjects will receive a single intravenous (IV) dose of study treatment (the HCl salt of Compound 1 or placebo) in addition to local standard of care treatment.

The injectable solution is a terminally sterilized solution composed of 25 mg/mL (adjusted free-base concentration) of the active pharmaceutical ingredient (API, Compound 1 HCl) in an aqueous, 10 mM citrate buffer with dextrose as a tonicity adjuster. A similar injectable solution was used in the nonclinical toxicology studies (10 mM citrate buffer and 289 mM dextrose in sterile water for injection).

After the PCI procedure, subjects will begin administration of an oral formulation (powder in capsule without excipients) of Compound 1 three times daily (TID) for the subsequent 30 days following the PCI procedure. On Day 2 and Day 30, subjects will undergo cardiac magnetic resonance imaging (CMR) for assessment of MVO (via late gadolinium-enhanced CMR). Echocardiogram (ECHO) for assessment of LV function will be performed on Day 2, Day 30, and Day 90.

Subjects should meet the following inclusion criteria to be eligible for enrollment into the study:
STEMI or NSTEMI patients undergoing PCI within 24 hours after diagnosis of ACS; and
Target lesions for PCI must appear suitable for stenting as confirmed on the diagnostic angiography. Acceptable lesions cannot be in the left main artery, or be a chronic total occlusion or in-stent restenosis.

The primary endpoint is the incidence of CMR-assessed MVO at two days post-PCI with Compound 1 versus placebo.

Secondary endpoints may include:
Incidence of CMR-assessed MVO at 30 days post-PCI with Compound 1 versus placebo
ECHO-assessed LV function based on the following parameters at 2, 30, and 90 days post-PCI with Compound 1 versus placebo:
   LV ejection fraction (LVEF)
   LV end-diastolic volume (LVEDV)
   LV end-systolic volume (LVESV)
   LV mass
Safety and tolerability of Compound 1.

Exploratory endpoints may include:
Incidence of MACE, defined as a composite of cardiac death, myocardial re-infarction, clinically driven myocardial revascularization, and hospitalization with discharge diagnosis of heart failure or coronary artery disease, during the 90 days post-PCI with Compound 1 versus placebo
Change in the following angiographic measures from pre-PCI to post-PCI (immediately after PCI) with Compound 1 versus placebo:
   Corrected thrombolysis in myocardial infarction frame count (cTFC)
   Thrombolysis in myocardial infarction flow grade (TFG)
   Thrombolysis in myocardial infarction myocardial perfusion grade (TMPG)
CMR-assessed myocardial response and LV function based on parameters such as the following at 2 and 30 days post-PCI with Compound 1 versus placebo:
   Infarct size (% of LV mass)
   Myocardial hemorrhage
   Myocardial edema
   Myocardial salvage index
   LVEF
   LVEDV
   LVESV

### Example 7—Clinical Trial 5

A phase 2, multicenter, randomized, double-blind, placebo-controlled study will be conducted to assess the safety, tolerability, PK, and efficacy of IV dosing of Compound 1 on microvascular obstruction (MVO) in subjects undergoing percutaneous coronary intervention (PCI). The study will be conducted in two stages (Stage A and Stage B), with each stage including a screening period of up to 14 days, a single dose of randomized study treatment (the HCl salt of Compound 1 or placebo) on Day 1, and a follow-up phone call 7 days (f 2 days) after administration of study treatment, for a total study duration of 6 to 24 days.

Study treatment will be administered following assessment of baseline angiographic measures, index of microcirculatory resistance (IMR) and additional coronary physiology indices, and blood sample collection (for assessment of peripheral serotonin concentration). Each subject will receive an intravenous (IV) single dose of study treatment. Assessment of IMR and additional coronary physiology indices will be repeated after administration of study treatment and before the start of the PCI procedure to obtain pre-PCI values. Immediately after completion of PCI, the following assessments will be performed to obtain post-PCI values: assessment of IMR and additional coronary physiology indices, angiographic measures, and collection of blood samples (obtained from the coronary ostium and coronary artery distal to the lesion for assessment of coronary artery serotonin concentration, as well as venous blood samples for peripheral serotonin concentration).

Stage A is an ascending single-dose study planned to consist of two cohorts. After treatment of each cohort in Stage A, a safety/tolerability assessment will be conducted by the Data and Safety Monitoring Board (DSMB) to determine whether dose escalation to the next dose level in Stage A or progression to Stage B can occur. One additional dose cohort may be explored in Stage A if deemed appropriate based upon data reviews of prior cohorts.

After conduct of the final safety review of Stage A, Stage B will begin. Stage B is a parallel-treatment group study planned to consist of a placebo group and two active treatment groups (doses of Compound 1 selected based on safety and tolerability data in Stage A). In Stage B, enrollment will consist of no more than 25% elective PCI subjects and randomization will be stratified by site and subject type (elective PCI or PCI for non-ST-elevation myocardial infarction [NSTEMI]/unstable angina [UA]).

Subjects should be treated with aspirin and an oral P2Y12 inhibitor (i.e., dual antiplatelet therapy [DAPT]) per site standard of care; however, subjects in Stage A may only be treated with clopidogrel as the P2Y12 inhibitor DAPT component and may not be treated with prasugrel or ticagrelor until after the subject has completed study participation.

The study treatment is an IV formulation containing the HCl salt of Compound 1 provided as 25 mg/mL strength (free base concentration). Subjects assigned to active treatment will receive one single dose in total as a 5 mL (diluted in saline) IV bolus administered in the forearm over duration of at least five minutes.

The dose to be administered to Cohort 1 of Stage A will be 20 mg (to be administered over a period of not less than 5 minutes). The dose administered in Cohort 2 is planned to be 40 mg. One additional dose cohort may be explored in Stage A if deemed appropriate based upon data reviews of prior cohorts. Selected doses from Stage A are planned to be investigated in Stage B of the study. Planned doses in Stage A and Stage B may be adjusted depending on the safety and tolerability results of previous cohort(s).

The primary endpoint is change in IMR from baseline to post-PCI with Compound 1, versus placebo.

Secondary endpoints may include:
Change from baseline to post-PCI with Compound 1 versus placebo for the following assessments:
   Coronary physiology indices (coronary flow reserve [CFR], fractional flow reserve [FFR])
   Angiographic measures (corrected thrombolysis in myocardial infarction frame count [cTFC], TFG, thrombolysis in myocardial infarction myocardial perfusion grade [TMPG])
   Myocardial injury markers (creatine kinase [CK], creatine kinase-myocardial band [CK-MB], cTn)
   Note: Post-PCI measure for CK, CK-MB, and cTn, is collected 4 to 6 hours after completion of the PCI procedure and not immediately after completion of the procedure.
The incidence of procedural myocardial injury with Compound 1 versus placebo defined as elevation of cTn values >99th percentile upper reference limit (URL) in subjects with normal Baseline values (≤99th percentile URL) or elevation of cTn by >20% of the baseline value in subjects with elevated cTn levels (>99th percentile URL)
Concentration of Compound 1 and active metabolites M1 and M2 prior to PCI and at selected post-PCI timepoints until discharge
Safety and tolerability of Compound 1

Subjects must meet all of the following inclusion criteria to be eligible for enrollment into the study:
Stable angina patients referred for elective PCI, or NSTEMI and UA patients undergoing PCI at least 12 hours after diagnosis for NSTEMI/UA. NSTEMI/UA patients are to be consistently hemodynamically stable until the time of PCI and have a thrombolysis in myocardial infarction (TIMI) Flow Grade (TFG) 3 on the diagnostic angiography
Target lesions for PCI must appear suitable for stenting as confirmed on the diagnostic angiography. The lesion must be located in a ≥2.75 mm diameter coronary artery; the lesion must also be ≥18 mm long and require the use of one or more stents that in total must be ≥20 mm long. Acceptable lesions cannot be in the left main artery or in a vein or arterial graft, or be a chronic total occlusion or in-stent restenosis. Two or more sequential lesions may be treated in the same artery, as long as they are treated in the same session and at least one of the lesions meets inclusion criteria
30 to 75 years of age, inclusive
Body mass index 18.0 to 40.0 kg/m2, inclusive

Subjects will be excluded from the study if they meet any of the following key exclusion criteria:
Planned or anticipated use of rotational atherectomy/ablation or shockwave therapies during the PCI procedure
Any history of stroke, head injury, seizure, intracranial bleeding, or intracranial aneurysm
Transient ischemic attack within the 6 months prior to Screening
History of major trauma, major surgery, and/or clinically significant hemorrhage within the last 6 months of screening
NSTEMI/UA with cardiac troponin (cTn) levels that are increasing (not stable or dropping), as shown by the two most recent measures after diagnosis of NSTEMI/UA and prior to randomization
Any ST-elevation myocardial infarction (STEMI) within 10 days of screening or STEMI within the target vessel territory within the last 6 months of screening (e.g., a patient with a NSTEMI because of a lesion in a diagonal may not be included if there is a history of anterior STEMI due to left anterior descending artery [LAD] lesion that occurred within the last 6 months)
Known history of heart failure
Vasculitis within the last 6 months of screening
Known contraindication or allergy to heparin (e.g., history of heparin-induced thrombocytopenia [HIT]), any antiplatelet agents (i.e., aspirin, P2Y12 inhibitors), or adenosine
Actively being treated with strong inhibitors or inducers of cytochrome P450 (CYP)3A4 or P-glycoprotein (examples include, but are not limited to, ketoconazole, itraconazole, clarithromycin, erythromycin, rifampicin, and verapamil). Intra-arterial use of verapamil for subjects undergoing radial artery PCI is permitted
Use of medications affecting the serotonin system (e.g., selective serotonin reuptake inhibitors [SSRIs], serotonin-norepinephrine reuptake inhibitors [SNRIs], atypical [second generation] antipsychotics possessing 5-HT2A pharmacology, or any other serotonergic agents possessing 5-HT2A pharmacology [e.g., the triazolopyridine antidepressant, trazodone]) within 14 days of check-in
Treatment with cangrelor or glycoprotein IIb/IIIa inhibitors within 24 hours of check-in
Need for chronic oral anticoagulant (e.g., warfarin or direct oral anticoagulants [DOACs], including direct oral thrombin inhibitors) for any condition (e.g., treatment of atrial fibrillation, deep vein thrombosis, or prosthetic cardiac valve) within 1 week of check-in
For subjects in Stage A ONLY: Use of prasugrel or ticagrelor within 14 days of check-in

### Example 8—Clinical Trial 6

A phase 1, open-label, parallel-cohort, fixed sequence study will be conducted to evaluate a food effect and drug-drug interactions (DDI) with Compound 1 in healthy adult subjects. Approximately 66 healthy adult subjects will be assigned to 1 of 3 cohorts (22 subjects each).

The PK profile of Compound 1, M1, and M2 will be assessed in each cohort for up to 3 to 5 days after each Compound 1 dose within the corresponding Treatment Period. Cohorts and treatments are defined below:

### Cohort 1 (Food Effect and Esomeprazole (ESO) DDI):

Subjects will be randomized (1:1) into one of two treatment sequences: A1:B:C (Sequence 1) or B:A1:C (Sequence 2). During Treatment Period 3, all subjects will be administered Treatment C. Subjects will remain in the study center for a domiciled period of approximately 13 days. Blood samples will be collected for PK analysis of Compound 1, M1, and M2 up to 3 days post each dose of Compound 1.

Treatments are defined below:
Treatment A1 (Reference): One 120 mg dose of Compound 1 (6×20 mg capsule) administered in the fasted state. PK sampling will continue for up to 3 days post dose.
Treatment B (Test): One 120 mg dose of Compound 1 (6×20 mg capsule) will be administered approximately 30 minutes after consumption of the FDA recommended high fat/high calorie breakfast. PK sampling will continue for up to 3 days post dose.
Treatment C (Test): In Treatment Period 3, 40 mg ESO (1×40 mg capsule) will be administered with regular diet qAM for 8 days (Day 7-Day 13), except for Day 10 where ESO will be administered in the fasted state. On Day 10, one 120 mg dose of Compound 1 (6×20 mg capsule) will be administered in the fasted state approximately 1 hour after ESO administration. Blood collection for PK will continue for up to 3 days post dose.

Study treatments and meals: All treatments will be administered in the morning. Treatment A1 will be administered following an overnight fist (i.e., no food or drink, except for water [excluding the 1 hour before and after dosing]) of at least 10 hours duration. Treatment B will be administered approximately 30-minutes after consumption of the FDA recommended high fat/high calorie breakfast. Food will be withheld up to 4 hours post administration of Compound 1 for both Treatment A1, Treatment B and Treatment C. For Treatment C, ESO will be administered 1 hour before regular morning meal on Days 7 to 9 and Days 11 to 13. On Day 10, ESO will be administered in the fasted state at least 1 hour prior to administration of Compound 1.

### Cohort 2 (Itraconazole (ITZ)/Fluconazole (FLU) DDI):

Subjects will be assigned to fixed treatment sequence A2:D:E (Sequence 1). Subjects will remain in the study center for a domiciled period of approximately 24 days. Blood samples will be collected for PK analysis of Compound 1, M1, and M2 up to 5 days post each dose of Compound 1, and for itraconazole (ITZ) or fluconazole (FLU) as appropriate. Treatments are defined below:
Treatment A2 (Reference): During Treatment Period 1, one 40 mg dose of Compound 1 (2×20 mg capsule) will be administered on Day 1 in the fasted state. Blood collection for PK will continue for up to 3 days post dose.
Treatment D (Test): During Treatment Period 2, FLU will be administered once daily (qAM) with breakfast (except for Day 7) as a loading dose of 400 mg on Day 4, followed by 200 mg on Day 5 to Day 12. On Day 7, a single 40 mg dose of Compound 1 in the fasted state will be administered 2 hours after FLU administration. Blood collection for PK will continue up to 5 days post Compound 1 dose. No treatments will be administered on Day 13 to Day 15.
Treatment E (Test): In Treatment Period 3, ITZ 200 mg (20 mL of 10 mg/mL oral solution) will be administered in the fasted state once daily in the morning (qAM) for 9 days (Day 16-Day 24). One 40 mg dose of Compound 1 (2×20 mg capsule) will be administered in the fasted state on the fourth day of ITZ treatment (Day 19) approximately 2 hours after ITZ administration. Blood collection for PK will continue to Day 24 (5 days post Compound 1 dose).

Study treatments and meals: All treatments will be administered in the morning. Treatments A2 (Compound 1) and E (ITZ) will be administered following an overnight fast (i.e., no food or drink, except for water [excluding the 1 hour before and after dosing]) of at least 10 hours. In Treatment D, FLU will be administered with normal morning meal except for Day 7. On Day 7, Compound 1 and FLU will be administered in the fasted state. All treatments will be administered within f 1 hour of dosing time established on Day 1 of Treatment Period 1. Following each Compound 1 administration, the fast will continue for at least 4 hours and for at least 1 hour following each FLU and ITZ administration.

Cohort 3 (rifampin (RIF) DDD:

Subjects will be assigned to a fixed sequence: A1:F. Subjects will remain in the study center for a domiciled period of approximately 20 days. Blood samples will be collected for PK analysis of Compound 1, M1, and M2 up to 5 days post each dose of Compound 1, and for rifampin (RIF) as appropriate.

Treatments are defined below:
Treatment A1 (Reference): In Treatment Period 1, one 120 mg dose of Compound 1 (6×20 mg capsule) will be administered on Day 1 in the fasted state. PK sampling will continue for up to 3 days post dose.
Treatment F (Test): In Treatment Period 2, RIF 600 mg (2×300 capsules) will be administered in the fasted qAM 10 days (Day 4 to Day 13). On Day 15, two days after the last RIF dose, one 120 mg dose of Compound 1 (6×20 mg capsule) will be administered in the fasted state. Collection of plasma for PK determinations will continue to Day 20 (5 days post Compound 1 dose).

Study treatments and meals: All treatments will be administered in the morning following an overnight fast (i.e., no food or drink, except for water [excluding the 1 hour before and after dosing]) of at least 10 hours. Compound 1 and RIF will be administered within f 1 hour of dosing time established on Day 1. Following Compound 1 administration, the fast will continue for at least 4 hours and for at least 1 hour following each RIF administration.

### All Cohorts:

Cardiodynamic electrocardiogram (ECG) extractions will be collected at scheduled time points.

The clinic will attempt to contact all subjects (including subjects who terminate the study early) using their standard procedures 7 (f 2) days after discharge to determine if any adverse events (AEs) have occurred since discharge or early termination.

### Test Product, Dose. and Mode of Administration:

Compound 1 is a 20 mg immediate-release capsule formulation composed of a size 2, hard-gelatin capsule containing Compound 1 HCl drug substance and no excipients.
In Cohort 1, 120 mg Compound 1 (6×20 mg capsule) will be administered as a single oral dose in the fasted or fed state with approximately 240 mL of water.
In Cohort 2, 40 mg Compound 1 (2×20 mg capsule) will be administered as a single oral dose in the fasted state with approximately 240 mL of water.
In Cohort 3, 120 mg Compound 1 (6×20 mg capsule) will be administered as a single oral dose in the fasted state with approximately 240 mL of water.

### Duration of Treatment:

The total study duration is up to 48, 59, and 55 days (f 1 day) for subjects in Cohorts 1, 2, and 3, respectively. The study includes a Screening Period (up to 28 days), a 13-day (Cohort 1), 24-day (Cohort 2) and 20-day (Cohort 3) domiciled treatment period, and a follow-up call 7 (f 2) days after discharge. Interacting Therapy/Reference Therapy, Dose, and Mode of Administration:

In Cohort 1 Treatment Period 3, 40 mg ESO (1×40 mg capsule) will be administered qAM for 7 consecutive days in the fed state (normal diet) with approximately 240 mL of water, except on Day 10 when it will be administered in the fasted state. Reference therapy will be Compound 1 (120 mg, 6×20 mg capsule), administered on Day 10 in the fasted state with approximately 240 mL water.

In Cohort 2 Treatment Period 2, 400 mg FLU loading dose (2×200 mg tablets) followed by 200 mg (1×200 mg tablet) will be administered in the fed condition (normal diet) qAM with approximately 240 mL water for 9 consecutive days, except on Day 7 when it will be administered in the fasted state. Reference therapy will be Compound 1 (40 mg, 2×20 mg capsule), administered with approximately 240 mL water in the fasted state on Day 7.

In Cohort 2 Treatment Period 3, 200 mg ITZ (20 mL of 10 mg/mL oral solution) will be administered qAM for 9 consecutive days in the fasted state with approximately 240 mL of water. Reference therapy will be Compound 1 (40 mg, 2× 20 mg capsule), administered with approximately 240 mL water in the fasted state on Day 19.

In Cohort 3 Treatment Period 2, 600 mg RIF (2×300 mg capsules) will be administered qAM for consecutive days in the fasted state with approximately 240 mL of water. Reference therapy will be Compound 1 (120 mg, 6×20 mg capsule), administered with approximately 240 mL water in the fasted state on Day 12.

Primary endpoints may include the following:
The following plasma PK parameters will be calculated for Compound 1, M1, and M2 in the absence and presence of food, ITZ, FLU, ESO and after multiple dosing with RIF:
   Area under the concentration-time curve from time 0 to last measurable concentration (AUC)
   Area under the concentration-time curve from time 0 to infinity (AUC_{0-∞})
   Maximum plasma concentration (Cₘₐₓ)
   Time to maximum plasma concentration (tₘₐₓ)
      Secondary endpoints may include the following:
Incidence of any treatment emergent adverse events (TEAEs)
Clinical laboratory values (serum chemistry, hematology, coagulation, and urinalysis), vital signs, ECG and physical examination
Additional plasma PK parameters of Compound 1, M1, and M2:
   Apparent total body clearance after the oral dose (CL/F), parent only
   Apparent volume of distribution after the oral dose (Vd/F), parent only
   Elimination half-life (t_{1/2})
   Mean residence time from time zero to the time of the last quantifiable sample (MRT₀₋ₜ), parent only
   Mean residence time from time zero extrapolate to infinity (MRT_{0-∞}), parent only
Metabolite to parent ratios for AUC and Cₘₐₓ will also be calculated
   Exploratory endpoints may include the following:
Plasma Concentrations/PK of ESO, ITZ, FLU and RIF, as appropriate
   Area under the concentration-time curve from time 0 to last measurable concentration (AUCₜ)
   Area under the concentration-time curve from time 0 to infinity (AUC_{0-∞})
   Maximum plasma concentration (Cₘₐₓ)
   Time to maximum plasma concentration (tₘₐₓ)
   C_{trough}, RIF only
The following urine PK parameters of Compound 1, M1, and M2 will be calculated, as appropriate:
   Amount of unchanged drug excreted in the urine collection interval from t₁ to t₂ (Aeti-t₂) (Fe)
   Renal clearance (CL_{R})—Compound 1 only

Safety assessments will include monitoring of adverse events (AEs), vital signs, clinical laboratory values, ECGs, and physical examinations.

Other uses of the disclosed methods will become apparent to those in the art based upon, inter alia, a review of this patent document.

1. A method of treating or preventing microvascular obstruction in an individual in need thereof, comprising:
administering parenterally to the individual a parenteral formulation comprising a first therapeutically effective amount of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof.

2. The method of claim 1, comprising:
a) administering parentally to the individual the parenteral formulation for a first period of time; and
b) after the administering parenterally, administering orally to the individual an oral formulation comprising a second therapeutically effective amount of Compound 1, or a pharmaceutically acceptable salt, hydrate, or solvate thereof, to the individual for a second period of time.

3. (canceled)

4. A method for preserving vascular integrity in an individual in need thereof, comprising:
a) administering parenterally to the individual a parenteral formulation comprising a first therapeutically effective amount of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to the individual for a first period of time; and
b) after the administering parenterally, administering orally to the individual an oral formulation comprising a second therapeutically effective amount of Compound 1, or a pharmaceutically acceptable salt, hydrate, or solvate thereof, to the individual for a second period of time.

5-7. (canceled)

8. The method of claim 1, wherein the individual is undergoing Percutaneous coronary intervention (PCI) for acute coronary syndrome (ACS).

9-11. (canceled)

12. The method of claim 8, wherein the parenteral formulation, is administered immediately prior to PCI.

13-17. (canceled)

18. The method of claim 1, wherein the parenteral formulation, is a sterilized solution comprising from about 1 mg/mL to about 50 mg/mL of an adjusted free-base concentration of Compound 1, or a pharmaceutically acceptable salt, hydrate, or solvate thereof.

19-23. (canceled)

24. The method of claim 1, wherein the parenteral formulation is administered intravenously.

25. The method of claim 1, wherein the parenteral formulation is administered by infusion.

26. (canceled)

27. The method of claim 2, wherein the administering orally comprises administering an oral loading dose and administering an oral maintenance dose.

28. The method of claim 1, wherein the first therapeutically effective amount of Compound 1, or a pharmaceutically acceptable salt, hydrate, or solvate thereof is about 5 mg, about 10 mg, about 15 mg, about 20 mg, about 25 mg, about 30 mg, about 35 mg, about 40 mg, about 45 mg, about 50 mg, about 55, g, about 60 mg, about 65 mg, about 70 mg, or about 75 mg.

29. (canceled)

30. The method of claim 1, wherein the parenteral formulation is administered over a period of about 5 minutes or less, about 4 minutes or less, about 3 minutes or less, au 2 minutes or less, or about 1 minute or less.

31. The method of claim 2, wherein the oral formulation is administered following percutaneous coronary intervention (PCI).

32. The method of claim 2, wherein the oral second therapeutically effective amount of Compound 1, or a pharmaceutically acceptable salt, hydrate, or solvate thereof is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85,_g, about 90 mg, about 95 mg, about 100 mg, about 105 mg, about 110, g, about 115 mg, about 120 mg, about 125 mg, about 130 mg, about 135 mg, about 140 mg, about 145 mg, abo 150_mg, au 155_mg, or about 160 mg.

33. The method of any claim 2, wherein the oral formulation is administered three times a day.

34. The method of claim 2, wherein the second period of time is at least one week.

35-38. (canceled)

39. The method of claim 1, wherein the Compound 1, or a pharmaceutically acceptable salt, hydrate, or solvate thereof, is an HCl salt of Compound 1.

40-43. (canceled)

44. The method of claim 1, wherein said method results in reduction in an incidence of a major adverse cardiac event (MACE).

45-47. (canceled)

48. A kit comprising:
a) parenteral formulation comprising a first therapeutically effective amount of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof;
b) an oral formulation comprising a second therapeutically effective amount of Compound 1, or a pharmaceutically acceptable salt, hydrate, or solvate thereof; and
g) instructions indicating the parental formulation and the oral formulation are for sequential use to prevent microvascular obstruction, to preserve vascular integrity, and/or to prevent a major adverse cardiovascular event (MACE) by preventing and/or treating microvascular obstruction (MVO) in an individual undergoing percutaneous coronary intervention (PCI).

49. (canceled)

50. A pharmaceutical formulation for parenteral administration comprising a sterilized solution comprising from about 1 mg/mL to about 25 mg/mL of an adjusted free-base concentration of 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof.

51-56. (canceled)

57. A method of safely administering 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof, to an individual in need thereof, comprising:
discontinuing administration of an inhibitor or inducer of CYP3A4, CYP3A5 and/or P-glycoprotein, or a proton pump inhibitor, to the individual;
not co-administering an inhibitor or inducer of CYP3A4, CYP3A5 and/or P-glycoprotein, or a proton pump inhibitor, to the individual;
co-administering a reduced dose of an inhibitor or inducer of CYP3A4, CYP3A5 and/or P-glycoprotein, or a proton pump inhibitor, to the individual; or
administering a reduced dose of the Compound 1, or a pharmaceutically acceptable salt, hydrate, or solvate thereof to the individual, when co-administered with an inhibitor or inducer of CYP3A4, CYP3A5 and/or P-glycoprotein, or a proton pump inhibitor; or
administering a lower dose of the Compound 1, or a pharmaceutically acceptable salt, hydrate, or solvate thereof and a lower dose of an inhibitor or inducer of CYP3A4, CYP3A5 and/or P-glycoprotein, or a proton pump inhibitor, to the individual.

58-61. (canceled)
